(19) United States
(12) Patent Application Publication
(10) Pub. No.: US 2009/0088624 A1
(43) Pub. Date: Apr. 2, 2009

(54) Methods and Apparatus for Diagnosing and Treating Aneurysms

(76) Inventor: Karen Nussbaumer, New Rochelle, NY (US)

Correspondence Address: WOOD, HERRON & EVANS, LLP, 2700 CAREW TOWER, 441 VINE STREET, CINCINNATI, OH, 45202, US

(21) Appl. No.: 12/161,268

(22) PCT Filed: Jan. 23, 2007
(86) PCT No.: PCT/US07/60898

§ 371 Date: Jul. 17, 2008

Related U.S. Application Data
(60) Provisional application No. 60/760,884, filed on Jan. 23, 2006.

(51) Int. Cl. A61B 5/055; A61B 8/13; A61B 6/03

(52) U.S. Cl. 600/411; 600/410; 600/437; 600/425; 600/427; 600/439

(57) ABSTRACT

A method of developing a diagnosis and prognosis for an aneurysm in order to determine a procedure for treating the aneurysm. The method includes determining a first diameter of a blood vessel at a first location proximal to an aneurysm, and determining a second diameter of the blood vessel at a second location. The second location incorporates at least a portion of the aneurysm. Next, the ratio of the second diameter to the first diameter is calculated. And, a prognosis of the aneurysm, and a determination of the treatment for the aneurysm, may then be determined based on the ratio.

### CROSS-REFERENCE TO RELATED APPLICATIONS

The present application is a 371 national phase of International Patent Application No. PCT/US07/60898, filed Jan. 23, 2007, and claims the benefit of U.S. Provisional Application Ser. No. 60/760,884, filed Jan. 23, 2006, the disclosures of which are incorporated by reference herein in their entireties.

### FIELD OF THE INVENTION

The present invention generally relates to treatment of aneurysms, and particularly relates to methods and apparatus for diagnosing, determining prognosis, and treating aneurysms, such as abdominal aortic aneurysms.

### BACKGROUND

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Proper functioning of the vascular system is essential for health and fitness. The vascular system, via blood, carries essential nutrients and gases to all living tissues and removes waste products for excretion. The vasculature is divided into different regions depending on the organ systems served. If vessels feeding a specific organ or group of organs are compromised, the organs and tissues supplied by those vessels are compromised and may fail. The failure of an organ or organs, or the failure of a blood vessel itself may even prove fatal.

One example of a condition that compromises a blood vessel is an aneurysm. An aneurysm is a localized, blood-filled dilation of a blood vessel or cardiac chamber caused by disease, such as arteriosclerosis, or weakening of the vessel or chamber wall. A “dilation,” as used herein, can be any expansion or dilatation of a particular blood vessel or cardiac chamber. Aneurysms, if not treated, may rupture. A ruptured aneurysm results in hemorrhage and is often fatal.

Aneurysms can form in any blood vessel, anywhere in the body, including the brain. However, those that form in veins are not life threatening, not routinely diagnosed, and not as likely to rupture as those that form in arteries. Most aneurysms occur in the aorta—the body's largest artery. The aorta, which resembles a garden hose in thickness, runs from the heart down the center of the chest and abdomen, eventually splitting into two arteries, one that serves each leg.

Aneurysms can develop anywhere along the aorta, but most occur in the section running through the abdomen (abdominal aortic aneurysms—AAAs). The rest occur in the section that runs through the upper chest (thoracic aortic aneurysms).

An aortic aneurysm is serious because—depending on its size—it may rupture, causing life-threatening internal bleeding. Each year, physicians diagnose approximately 200,000 people in the United States with AAA. Of those 200,000, nearly 15,000 may have AAA threatening enough to cause death from a ruptured aneurysm if not treated.

Fortunately, when diagnosed early, AAAs can be treated, or even cured, with highly effective and safe treatments. Surgery after rupture isn't always successful. Surgery before rupture is more effective, when the aortic aneurysm is detected in time.

Thus, aneurysms are classified based on the blood vessel in which they appear. For example, in the abdomen, one particular type of aneurysm is the AAA. Current criteria state that a localized dilation of the abdominal aorta that is greater than 3.0 centimeters (cm) is considered an AAA. Aortic diameters measuring under 3.0 cm are not considered aneurismal. Slightly enlarged aortas are defined as ectatic. Current criteria states that the aortic diameter measurement for an ectatic aorta is less than 3.0 cm. Some references consider a 3.0 cm diameter to be aneurismal, while others consider it ectatic.

AAAs generally result from a degenerative process involving the aortic wall. The deterioration and weakening of the vessel wall can be caused by a number of factors including smoking, high blood pressure, high cholesterol, and certain diseases like Marfan syndrome and Ehlers-Danlos syndrome, for example. AAAs are most commonly located infrarenally, although other possible locations are suprarenal and pararenal. The vast majority of aneurysms are asymptomatic. The risk of rupture is high in a symptomatic aneurysm, which is therefore considered an indication for surgery. Symptoms may include low back pain, flank pain, abdominal pain, groin pain, or pulsating abdominal mass. The complications include rupture, peripheral embolization, acute aortic occlusion, and aortocaval or aortoduodenal fistulae. On physical examination, a palpable abdominal mass can be noted. Bruits can be present in case of renal or visceral arterial stenosis.

AAAs are commonly divided according to their size. As mentioned above, current data suggests that an aortic diameter of 3.0 cm can be considered ectatic, i.e., slightly enlarged. And current literature suggests that if an aortic diameter exceeds 5.0 cm, the AAA is considered to be large, while those less than 5.0 cm are considered small.

Recent literature indicates that women's aneurysms rupture at a rate up to five times greater than men's aneurysms. There is no concrete explanation for this increase in rupture at this time. And so, currently there is no way to accurately predict the likelihood of rupture of smaller aneurysms. Further, some aneurysms are completely ignored due to their small size as physicians deem them to be merely ectatic.

Presently, the diagnosis of an AAA includes two steps: (1) diagnosis and (2) assessment of severity, with the assessment of severity being based, at least in part, on the size of the AAA. As most AAAs are asymptomatic, their presence is usually revealed during an abdominal examination for another reason. It is common for physicians to order an ultrasound to rule out alternate abdominal pathology. During the course of the ultrasound, the incidental diagnosis of AAAs can be made. There are, however, AAA ultrasound screenings that are available for asymptomatic patients. Ultrasonography can provide the initial assessment of the size and extent of the aneurysm. Correlative and/or alternative examinations may include CT, MRI, and special modes thereof, like CT/MR angiography.

Ultrasound examination of the abdominal aorta includes, among other measurements, a transverse measurement of the proximal, mid and distal aorta, as is well known to those skilled in the art. The average measurement of a normal aorta is 2.5 cm proximal, 2.0 cm mid and 1.5 cm distal. As described above, typically, an aortic diameter measurement exceeding 3.0 cm is considered aneurismal. The concern regarding the treatment of AAA is whether to intervene to repair the aneurysm (e.g., by surgery) or to monitor the rate of aneurismal growth periodically. The decision on how to proceed with treating an aneurysm is generally decided by the size of the aneurysm.

When evaluating the risk of AAA rupture, a measurement of the maximal diameter of the aneurysm is taken into consideration. Generally, AAAs measuring 5.0 cm, or greater (i.e., large AAAs), in diameter are treated. Generally, smaller AAAs, measuring less than 5.0 cm in diameter, are monitored periodically.

However, the 5.0 cm diameter criterion does not provide sufficient information pertaining to the amount of aortic dilation in all cases. Although the 5.0 cm diameter criterion is a predominant method for choosing between intervention or monitoring aneurismal progress, the rupture rate of the AAA remains much greater in women, as opposed to men, even using this criterion. Thus, a new system of diagnosing, and developing prognoses, in order to decide on the proper treatment of AAAs, and other aneurysms, that is effective in both men and women, is needed.

### SUMMARY

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

The present invention is based on the recognition by the inventor that the increased rate of rupture of aneurysms in women might be because blood vessel dilation is actually much greater in many females as compared to males, due to women's anatomically smaller vessel size. As described above, in the case of AAAs, the average normal aortic diameter is considered to be 2.0 cm, with a dilation of greater than 3.0 cm in diameter being classified as an aneurysm, and a diameter of 5.0 cm generally thought to require intervention, which is generally any treatment other than monitoring the progression of the aneurysm. However, these numbers will only hold true for those persons having a normal aortic diameter of 2.0 cm. Thus, when a person has a smaller native aorta, i.e., measuring less than the typical used 2.0 cm criterion, the AAA of that person may actually be much greater, and the patient may be a strong candidate for intervention, even though the AAA is less than 5.0 cm in diameter. However, this would not likely be recognized by current protocols, thereby leaving that individual at greater risk for rupture of the aneurysm and the dire consequences that follow. For example, if a person has a 2.9 cm dilation of the abdominal aorta and a native average aortic diameter of 1.1 cm, the 2.9 cm measurement would not even be considered an aneurysm at all by present standards. The “native” aorta is a part of the aorta that is unaffected by aneurismal dilation. Thus, one aspect of the present invention arises from the recognition that an aneurysm can occur even if it is less than the present 3.0 cm standard. Rather, the criteria of an aneurysm should be any abnormal dilation of a blood vessel, and thus the criteria of an AAA should be any abnormal dilation of the abdominal aorta.

Thus, another aspect of the present invention includes a method of diagnosing and developing a prognosis for an aneurysm in order to determine how to proceed in treating the aneurysm. The method eliminates the drawbacks of currently used methods, which rely predominantly on measurements of the aneurysm itself, by developing a ratio of the size of the aneurysm to the size of the blood vessel so that the method works successfully for all individuals, regardless of the diameter of the native aorta. This method includes determining a first diameter of a blood vessel at a first location proximal to an aneurysm, and determining a second diameter of the blood vessel at a second location, the second location incorporating at least a portion of the aneurysm. In one embodiment of the method, in the case of an AAA as an example, the first diameter is measured at an uninvolved part of the aorta proximal to an aneurysm, and the second diameter is measured at the maximal diameter of the aneurysm. The ratio of the second diameter to the first diameter (i.e., aneurysm to blood vessel) is then calculated by dividing the first diameter into the second diameter. A prognosis for the aneurysm can then be determined based on that ratio. In particular, a prognosis can be made as to whether the likelihood of rupture of the aneurysm is sufficient to warrant invasive intervention, or whether simply monitoring the progression of the aneurysm will suffice.

The use of such an aneurysm dilation ratio [referred to herein as the Nussbaumer Aneurysm Dilation Ratio (NADR)] involves not only the measurement of the maximal diameter of the aneurysm (which is the predominant criterion of present protocols, as described in the Background), but also the measurement of the diameter of the native, uninvolved blood vessel (e.g., the abdominal aorta). The NADR thus can accurately assess the risk of aneurysm rupture by calibrating the relative amount of dilation of the aorta in the form of a ratio.

Thus, the proposed NADR includes, in one embodiment, the measurement of the maximal diameter of the aneurysm (as the second diameter), and the diameter of the native, uninvolved aorta (as the first diameter) proximal to the aneurysm. As used here, “proximal” means above the aneurysm. Thus, if the aneurysm is in the mid-aorta, for example, the native measurement taken proximal to that will be taken between the proximal and mid-aorta. If the aneurysm is in the distal aorta, for example, the native measurement will be taken proximal to that, i.e., between the mid- and distal aorta. This measurement is generally taken as close as possible to the aneurysm. The NADR is used to assess the risk of rupture by calibrating the aorta's relative amount of dilation in ratio form. A 5.0:2.0 ratio (average diameter currently indicated for AAA surgical repair: average mid-aorta diameter) is considered the baseline ratio. This ratio is calculated from the known 5.0 cm aneurismal diameter criterion for proceeding with intervention when a blood vessel has a diameter of 2.0 cm. Thus, a NADR of 2.5 (i.e., 5.0 cm/2.0 cm) or greater, for example, may correlate to an increased rate of rupture of AAAs, and thus to surgical treatment. Smaller aneurysms that would be monitored under current protocols may now be considered for surgical repair, and aneurysms greater than 5.0 cm may now be monitored if they do not warrant surgical repair due to the larger size of the native aorta.

For example, a larger man (or woman) may have a normal, native, mid aorta size of 2.5 cm. If this patient has a 5.0 cm aneurysm, using the NADR criteria, this patient would have a ratio of 2.0. According to NADR criteria, this patient would not be a candidate for surgery. The risks of surgery for this patient at this time would outweigh the likelihood of rupture. Periodic monitoring, using the NADR criteria, would be suggested. This explains why some aneurysms do not rupture at 5.0 cm, or even greater, diameters.

Thus, the NADR provides an effective method for evaluating aneurysms (such as AAAs) and is a better predictor of aneurysm rupture than the current aneurysm diameter criteria. In addition, the exemplary ratio of 2.5 or greater will prove to be a better indicator of the need for intervention (which may include surgery), while an exemplary ratio of less than 2.5 will prove to be a better indicator of the need for periodic monitoring. The NADR can be used with any modality—including ultrasound, computed tomography and magnetic resonance imaging—when aortic measurements can be obtained. Modalities such as ultrasound are advantageous in that they are cost-effective, noninvasive, efficient, and accurate. Further, ultrasound includes noninvasive, “real-time” evaluation of blood flow hemodynamics and pathology that greatly benefits the interpreting physician, as well as the patient. Further, vascular ultrasound exams can be used to evaluate nearly any artery and vein.

Thus, in another aspect, the present invention may include an apparatus for use in diagnosing and developing a prognosis for an aneurysm. Such an apparatus may include a housing and a transmitter operatively connected to the housing for directing x-rays or electrical, mechanical, magnetic, or sound waves or pulses in a direction away from the transmitter and into a patient body toward a blood vessel of interest. The apparatus may further include a receiver operatively connected to the housing for receiving the rays, waves, or pulses reflected from a blood vessel in the patient body. These received rays, waves, or pulses may then be converted into an image of the blood vessel and aneurysm. For example, the receiver may be a transducer that is operable for receiving reflected waves or pulses and generating an image of the blood vessel from the reflected waves or pulses. Or the apparatus may include a controller operatively connected to the receiver. The receiver sends the information from the reflected rays, waves, or pulses to the controller, which then generates an image of the blood vessel and aneurysm from the reflected waves or pulses. The image may appear on a display screen (such as on a monitor). Additionally, the apparatus may be adapted to measure a first diameter of the imaged blood vessel at a first location proximal to an aneurysm and a second diameter of the imaged blood vessel at a second location incorporating at least a portion of the aneurysm. The NADR may then be calculated using these measurements.

Various features discussed below in relation to one or more of the exemplary embodiments may be incorporated into any of the above-described aspects of the present invention alone or in any combination. Again, the brief summary presented above is intended only to familiarize the reader with certain aspects and contexts of the present invention without limitation to the claimed subject matter.

### BRIEF DESCRIPTION OF THE FIGURES

Various features, aspects, and advantages of the present invention will become better understood when the following detailed description is read with reference to the accompanying figures in which like characters represent like parts throughout the figures, wherein:

FIG. 1 is a perspective view of a blood vessel of an average diameter exhibiting an aneurysm.

FIG. 2 is a perspective view of a blood vessel of a smaller-than-average diameter exhibiting an aneurysm.

FIG. 3 is a schematic of use of an apparatus to image a blood vessel to determine measurements of the blood vessel showing transmission of waves or pulses toward a blood vessel to be imaged.

FIG. 4 is schematic of use of an apparatus to image a blood vessel to determine measurements of the blood vessel showing reflection of waves or pulses from a blood vessel to be imaged.

### DETAILED DESCRIPTION OF EXEMPLARY EMBODIMENTS

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

One aspect of the present invention includes a method of diagnosing and developing a prognosis for an aneurysm in order to determine how to proceed in treating the aneurysm. The method eliminates the drawbacks of currently used methods, which rely predominantly on measurements of the aneurysm itself, by developing a ratio of the size of the aneurysm to the size of the blood vessel so that the method works successfully for all individuals, (i.e., both males and females). This method includes determining a first diameter of a blood vessel at a first location proximal to an aneurysm, and determining a second diameter of the blood vessel at a second location, the second location incorporating at least a portion of the aneurysm, and in certain embodiments the second location incorporating the maximal diameter of the aneurysm. As an example, in an exemplary embodiment in the case of the AAA, the first diameter is measured at an uninvolved part of the aorta proximal to an aneurysm, and the second diameter is measured at the maximal diameter of the aneurysm. The ratio of the second diameter to the first diameter (i.e., aneurysm to blood vessel) is then calculated by dividing the first diameter into the second diameter. A prognosis for the aneurysm can then be determined based on that ratio. In particular, a prognosis can be made as to whether the likelihood of rupture of the aneurysm is sufficient to warrant invasive intervention, or whether simply monitoring the progression of the aneurysm will suffice as a treatment.

Those of skill in the art will recognize that an AAA is not the only aneurysm that may be diagnosed, prognosed, and treated by the method of this aspect of the present invention, but that any other aneurysm may be diagnosed, prognosed, and treated by the same method. Further, those of skill in the art will recognize that any particular locations for an aneurysm described herein, such as a particular example of an AAA, does not necessarily mean that the method can only be used with aneurysms appearing particularly in that location. While herein, the AAA may be referenced as normally appearing just before the iliac bifurcation, the AAA may actually appear anywhere along the abdominal aorta. And thus, as will be recognized by those of skill in the art, the method of the present invention may be adapted to use on particular aneurysms, regardless of where they appear along a particular blood vessel.

Further, as used herein, “intervention” describes any treatment for an aneurysm other than monitoring progression of the aneurysm. Such intervention may include very invasive treatments such as surgery to repair the aneurysm (e.g., open abdominal or open chest repair—wherein the aneurysm is removed and the section of aorta is replaced with an artificial graft made of material such as Dacron® or Teflon®), to some less invasive treatments. For example, endovascular surgery is one such less invasive treatment. In endovascular surgery, a synthetic graft is attached to the end of a catheter that is inserted through an artery into a patient's leg and threaded up into the aorta. The graft—generally a woven tube covered by a metal mesh support—is deployed at the site of the aneurysm and fastened in place with small hooks or pins. The graft reinforces the weakened section of the aorta to prevent rupture of the aneurysm.

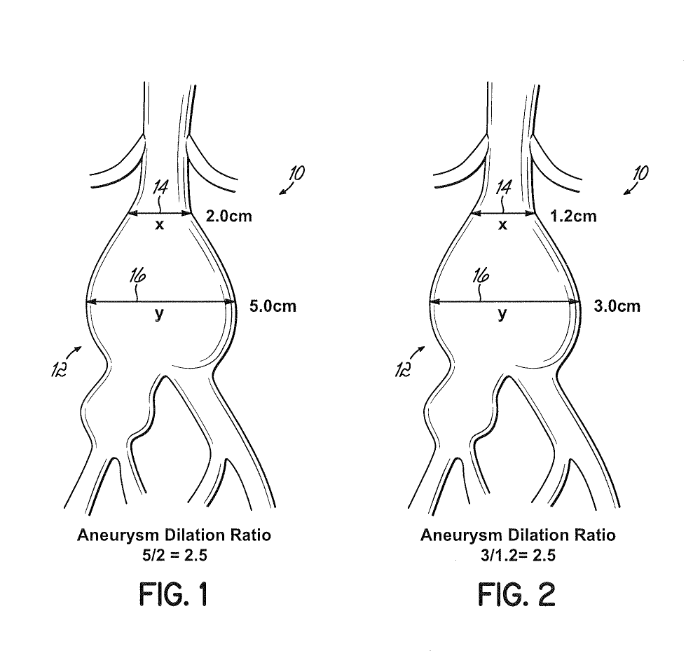

Referring now to FIGS. 1 and 2, a blood vessel 10 having an aneurysm 12 is shown. The blood vessel exhibits a first diameter (“X”) 14 at the mid-portion of the blood vessel, and a second diameter (“Y”) 16 at a second location of the blood vessel 10. This second portion of the blood vessel 10 includes the aneurysm 12, and the measurement of second diameter 16 is taken transverse to the aneurysm 12. An NADR of Y:X can be determined with the ratio thereof being Y divided by X. Thus, the ratio is a numerical value indicative of the treatment to be used. For example, as shown in FIG. 1, X=2.0 cm and Y=5.0 cm for a NADR of 5.0 cm/2.0 cm, which equals 2.5. Further, FIG. 2 shows X=1.2 cm and Y=3.0 cm, for an NADR of 3/1.2, which equals 2.5. Thus, using the NADR method, intervention could be the recommended treatment in the cases shown in both FIG. 1 and FIG. 2. Current protocols, however, would have only suggested intervention for FIG. 1, and not for the case of FIG. 2. The NADR, then, is more accurate in diagnosis, prognosis, and treatment than other previously and presently used protocols.

In order that the method should be noninvasive in certain embodiments, the first diameter of the blood vessel and second diameter of the aneurysm are not measured directly. Rather, the first diameter and second diameter may be determined using any of various apparatus that are suitable for imaging structures internal to a patient body without direct exposure of those structures. Examples of such apparatus include ultrasound apparatus, CT apparatus, and MRI apparatus.

Thus, in performing certain embodiments of the method, measurements of images of the blood vessel and aneurysm are taken. The measurements of these images correspond to the actual measurements of the blood vessel and aneurysm, and so the measurements of the blood vessel and aneurysm (i.e., the first and second diameters) can be determined. Stated another way, the NADR calculated from images of the blood vessel and aneurysm will equal the NADR of the actual blood vessel and aneurysm, thereby allowing for accurate diagnosis, prognosis, and treatment. Thus, measuring the first diameter of the blood vessel and measuring the second diameter of the aneurysm includes reviewing images of the blood vessel and aneurysm, and measuring a first diameter and a second diameter of the images of the blood vessel and aneurysm, in order to determine the first diameter and the second diameter of the blood vessel and aneurysm, respectively. In doing so, it will be recognized by those skilled in the art that the measurements of the images of the blood vessel and aneurysm may not correspond exactly to the exact measurements of the blood vessel itself and the aneurysm itself (were such direct measurements to be taken). However, the ratio to be calculated using the first diameter of the blood vessel and the second diameter of the aneurysm will be the same as if direct measurements were taken, and so the measurements of the images of the blood vessel and aneurysm can be considered to be adequate for determining the relevant diameters of the blood vessel itself and the aneurysm itself.

In order to create such images to determine the first diameter of the blood vessel and the second diameter of the blood vessel (at the aneurysm), the method further includes directing, for example, x-rays or electrical, mechanical, magnetic, or sound waves or pulses from a point exterior to a patient body into the patient body and toward a blood vessel of interest. There are several exemplary apparatus that may be sufficient to direct such x-rays or electrical, mechanical, magnetic, or sound waves or pulses into the patient body. For example, such apparatus may include an ultrasound apparatus, a CT apparatus, or a MRI apparatus.

As the rays, waves, or pulses are directed into the patient body via a transmitter, they can be directed toward the particular blood vessel of interest, and including the aneurismal portion thereof. When the rays, waves, or pulses reach the blood vessel, they will be reflected therefrom back toward a receiver, which may be a transducer, and converted into an image.

The method then includes reviewing the image and measuring a first diameter of the blood vessel and a second diameter of the image of the blood vessel at the aneurismal portion thereof, in order to determine the first diameter of the blood vessel and the second diameter of the blood vessel at the aneurismal portion thereof, as described above. In particular, once the image is processed, it can be visualized on a monitor and/or display screen. Thus, the measurements of the maximal diameter of the aneurysm (second measurement) and the uninvolved blood vessel diameter (first measurement), which is taken proximal to the second measurement, can be manually or electronically taken and the ratio can be determined manually or automatically via computer software or other apparatus.

Thus, once measurement values for the first diameter (diameter of the blood vessel) and second diameter (diameter of the aneurysm) have been obtained, a ratio of the second diameter to the first diameter may be obtained by dividing the measurement value for the second diameter by the measurement value for the first diameter. The resulting ratio can be used to assess the severity of the aneurysm (i.e., the prognosis) in order to ultimately determine whether to treat the aneurysm in a conservative manner by monitoring the progress of the aneurysm, or to treat by performing surgery, or otherwise intervening to repair the blood vessel.

Thus, the calculated ratio can be compared to a standard ratio that is developed based on a particular blood vessel being examined, the standard ratio reflecting the diameter of an aneurysm as compared to an average diameter blood vessel, which warrants treatment by intervention or treatment which may include surgery. If the calculated ratio is greater than or equal to the standard ratio, then the aneurysm may be assessed as likely to rupture such that intervention or treatment, which may include surgery, is warranted. However, if the calculated ratio is less than the standard ratio, then progression of the aneurysm may be monitored, thereby preventing an unnecessary intervention. As will be recognized by those skilled in the art, the diameters of blood vessels vary depending upon the particular blood vessel being examined.

As described above, one particular type of aneurysm is the AAA. The average measurement of the normal aorta is 2.5 cm proximal, 2.0 cm mid and 1.5 cm distal. Typically, an aorta diameter measurement exceeding 3.0 cm is considered aneurismal. Generally, AAAs measuring greater than 5.0 centimeters (cm) in diameter are surgically repaired and smaller aneurysms, measuring less than 5.0 cm in diameter, are monitored periodically. A 5.0:2.0 ratio (average diameter indicated for AAA surgical repair:average mid-aorta diameter) is considered for the baseline ratio value. Thus, a NADR of 2.5 or greater will likely correlate to an increased rate of rupture of AAAs. Smaller aneurysms that once would have been monitored now may be considered for surgical repair, and aneurysms greater than 5.0 cm may now be monitored, due to the size of their native aorta.

Thus, the NADR is an effective method for evaluating AAAs and a better predictor of AAA rupture than the current diameter criteria. In addition, the ratio value of 2.5 or greater will prove to be a better indicator of the need for surgical intervention, while a ratio value of less than 2.5 will prove to be a better indicator of the need for periodic monitoring. The following Tables 1, 2, and 3 provide an example of the use of the NADR in assessing the severity of AAAs.

TABLE 1

| Case Number | 1 | 2 | 3 | 4 |
|---|---|---|---|---|
| Maximal Aneurysm Diameter | 3.0 cm | 3.0 cm | 3.0 cm | 3.0 cm |
| Native Aorta Diameter | 1.0 cm | 1.5 cm | 2.0 cm | 2.5 cm |
| Nussbaumer Aneurysm Dilation | 3 | 2 | 1.5 | 1.2 |
| Ratio (NADR) | | | | |

TABLE 2

| Case Number | 1 | 2 | 3 | 4 |
|---|---|---|---|---|
| Maximal Aneurysm Diameter | 4.0 cm | 4.0 cm | 4.0 cm | 4.0 cm |
| Native Aorta Diameter | 1.0 cm | 1.5 cm | 2.0 cm | 2.5 cm |
| Nussbaumer Aneurysm Dilation | 4 | 2.7 | 2 | 1.6 |
| Ratio (NADR) | | | | |

TABLE 3

| Case Number | 1 | 2 | 3 | 4 |
|---|---|---|---|---|
| Maximal Aneurysm Diameter | 5.0 cm | 5.0 cm | 5.0 cm | 5.0 cm |
| Native Aorta Diameter | 1.0 cm | 1.5 cm | 2.0 cm | 2.5 cm |
| Nussbaumer Aneurysm Dilation | 5 | 3.3 | 2.5 | 2 |
| Ratio (NADR) | | | | |

As can be seen from Tables 1-3 and the previous discussion, current protocols for assessing the severity of AAAs and determining treatments therefore (based on the 5.0 cm diameter criterion), would suggest intervention to repair the abdominal aorta in each of the 4 cases shown in Table 3 (as each of those includes a maximal aneurysm diameter of 5.0 cm). The cases in Tables 1 and 2 only exhibit maximal aneurysm diameters of 3.0 cm and 4.0 cm, respectively, and so current protocols would not suggest intervention as the desired treatment in any of those cases.

However, using the method of the NADR, only three of the four cases listed in Table 3 would actually be strong candidates for intervention (i.e., the cases having NADRs of 5, 3.3, and 2.5). The fourth case, having an NADR of 2, would not be a strong candidate for intervention, and so using the presently disclosed method, a more conservative treatment of monitoring the progression of the AAA would be determined. Previous protocols would have suggested an unnecessary intervention being performed in the fourth case, along with attendant costs and possible dangers to the patient, which are present in any intervention, such as surgical procedures. The NADR avoids such an unnecessary intervention.

Further, current protocols would not suggest surgical repair for any of the eight cases shown in Tables 1 and 2. However, using the method of the NADR, three of those eight cases would actually be strong candidates for intervention, as they are at high risk for a rupture of the AAA. Case 1 of Table 1 has a NADR of 3, and Cases 1 and 2 of Table 2 have NADRs of 4 and 2.7, respectively. Using current protocols, the patients reflected by these cases would not have intervention to repair the abdominal aorta, thereby risking rupture, hemorrhage, and possibly death. The NADR, however, would more accurately predict the severity of these AAAs, thereby allowing one to proceed with a treatment of intervention, such as surgical repair, thereby avoiding the potentially serious ramifications of delaying intervention in these cases.

Thus, the method further includes determining a treatment for the aneurysm based on the prognosis provided by the NADR. As above, this treatment may be intervention, such as to repair the blood vessel, or may be monitoring the progression of the aneurysm.

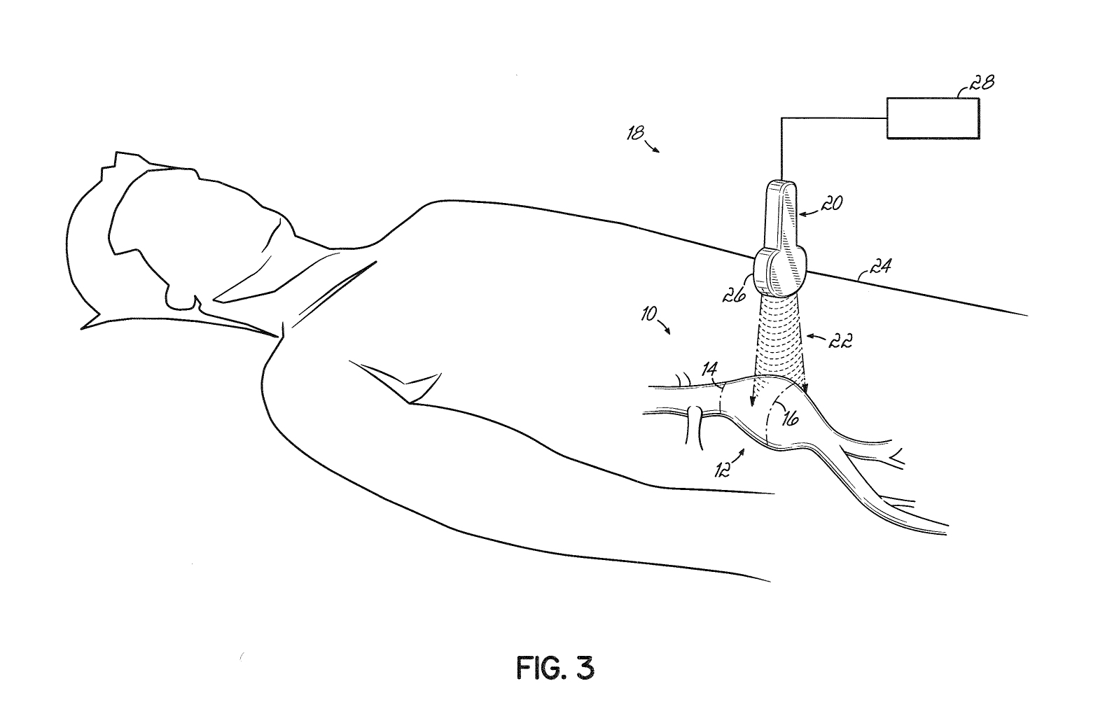

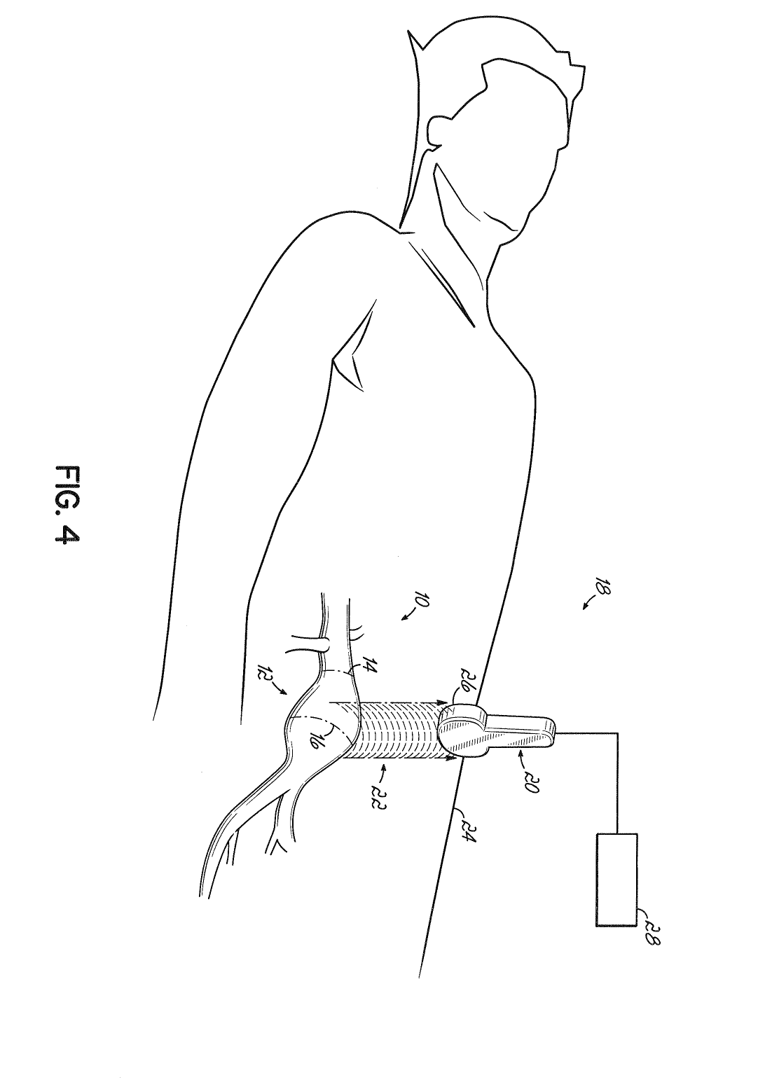

As described above, the method may not include actual, direct measurement of the blood vessel and aneurysm at issue. Rather, it may include obtaining an image of the blood vessel and aneurysm at issue, and determining a measurement of those images that corresponds to the actual measurement of the blood vessel such that the NADR can be determined. Thus, in another aspect, and referring now to FIG. 3, the present invention may include an apparatus 18 for use in diagnosing and developing a prognosis for an aneurysm 12. Such an apparatus 18 may include a housing (not shown) and a transmitter 20 operatively connected to the housing for directing x-rays or electrical, mechanical, magnetic, or sound waves or pulses 22 in a direction away from the transmitter 20 and into a patient body 24. The apparatus 18 may further include a receiver 26 operatively connected to the housing for receiving the x-rays, waves, or pulses 22 reflected from a blood vessel 10 in the patient body 24. Thus, the apparatus of the illustrated embodiment includes a transmitter 20 and receiver 26; these may be in the same housing, such as a probe housing. The apparatus 10 may further include a component that can convert the received rays, waves, or pulses 22 into an image of the blood vessel and aneurysm. This component may be a transducer (which includes the receiver 26), for example. Or, as another example, this component may be separate from the receiver 26, such as a controller 28 operatively connected to the receiver 26. Additionally, the controller 28 may be adapted to measure a first diameter 14 of the imaged blood vessel at a first location proximal to an aneurysm 12 and a second diameter 16 of the imaged blood vessel 14 at a second location incorporating a portion of the aneurysm 12.

In certain embodiments, the controller may be adapted to calculate the ratio (i.e., the NADR) of the second diameter to the first diameter. Further, in certain embodiments, the controller may be adapted to display the NADR, a prognosis of the aneurysm, and/or a suggested treatment based on the ratio (i.e., the NADR) on the display screen.

As an example, one apparatus that may be used in aspects of the present invention is an ultrasound apparatus, as described above. More specifically, medical ultrasonography is an ultrasound-based diagnostic imaging technique used to visualize muscles and internal organs, their size, structures and any pathological lesions by creating images of the same.

Ultrasound imaging systems have become an important diagnostic tool in many medical specialties. One important advantage of an ultrasound imaging system is real-time scanning. For example, an ultrasound imaging system can produce images via a transmitter/transducer. These images can be produced at a rate that allows a sonographer to scan internal organs or discern motion (such as blood flow) within a body, with real-time, visual feedback. This allows the sonographer to examine structures of interest and to modify the examination in real-time, thereby improving diagnostic quality. Currently two types of arrangements of transducers are used for ultrasound imaging systems.

One arrangement includes a single transducer or an annular array of transducers. Ultrasound imaging systems using this arrangement of transducers rely on mechanical motion of a probe (i.e., the transmitter) to direct an acoustic beam over a region of interest.

A second arrangement of transducers includes an array of transducers, which is activated by electronic circuits that produce electronically induced time delays in the transducer acoustic outputs. These time delays induce measurable phase delays, which cause the acoustic beam produced by the transmitter to be steered and/or focused.

As is known in the art, transducers in an ultrasound probe can be arranged in a one-dimensional (1-D) array, a one-and-a-half-dimensional (1.5-D) array, or a two-dimensional (2-D) array. In a 1-D array, transducers are generally disposed in the lateral direction, with a single row of transducers in the elevation direction. Conventional phase linear arrays and curved arrays are generally considered 1-D transducer arrays.

In a 1.5-D array, transducers are mounted in both the lateral and elevation directions, but control and data electrical connections are symmetrically connected about the elevation center so that an acoustic beam produced by a 1.5-D array can only be steered in the lateral direction. In a 2-D array, transducers are arranged in both the lateral and elevation directions, with electrical connections providing both transmit/receive control and excitation signals to transducers arranged in both directions. An acoustic beam produced by a 2-D array can be steered and focused in two dimensions.

Sonographers can obtain images of a region within a body by properly positioning an ultrasound transmitter/transducer against the body. In order to obtain images having diagnostic value, the sonographer may have to manipulate the position of the probe by moving the probe with respect to the patient.

The above description of ultrasound technology, in general, is widely known by those skilled in the art. This ultrasound technology can also be used to perform the necessary measurements of the blood vessel and aneurysm, such as AAAs, in order to calculate the NADR. In particular, the ultrasound apparatus may include software designed to measure the relevant measurements, and then automatically calculate the NADR, or can include software that allows the sonographer to make the relevant measurements. The measurements may then be entered on the apparatus, which automatically calculates the NADR, or alternatively, the sonographer may make the necessary calculation of the NADR.

In use, in one embodiment, an ultrasound probe (i.e., transmitter/transducer) is placed just below the sternum of a patient in a transverse plane. This is the general location for the proximal portion of the aorta. A diameter measurement is taken by pressing a cursor button on the ultrasound apparatus. This will bring a first caliper on a display screen of the apparatus. The first caliper can be manipulated by a user to move on the display screen and is placed to one side of the image of the blood vessel, (e.g., on the outer wall of the image of the blood vessel). By pressing a “set” key (sometimes termed as a “freeze” key or other), the first caliper will lock in place on the screen. Then the cursor button is pressed again, and a second caliper appears on the display screen. This second caliper is placed on a side of the image of the vessel opposite the first caliper. The set key is then pressed to lock the second caliper in place. The ultrasound machine can then be used to calculate the measurement between the first and second calipers. This procedure is repeated for proximal, mid- and distal aorta as well as the aortic bifurcation into the iliacs. This procedure is also repeated to measure the aneurysm itself. Typically, the aneurysm occurs distally, just before the area of bifurcation. This allows for the mid-aorta measurement to be used for the ratio. If the solo mid-aorta measurement is not clearly seen, any measurement of the normal aorta between the mid- and distal aorta can be used, but it should be as close as possible to the aneurysm. Again, it will be recognized by those skilled in the art that the measurements of the images of the blood vessel may not correspond exactly to the exact measurements of the blood vessel itself (were such direct measurements to be taken). However, the ratio to be calculated using the first diameter and the second diameter will be the same as if direct measurements were to be taken, and so the measurements of the images of the blood vessel can be considered to be adequate for determining the relevant first and second diameters of the blood vessel itself.

It will be understood by those skilled in the art that the above description of the use of the measurements and calculation of the NADR with ultrasound technology is merely one exemplary embodiment of how the method of the present invention might be performed. Those skilled in the art will recognize that other diagnostic tools, such as CT and MRI, may also be used to measure and calculate the NADR.

As various changes could be made in the above-described aspects and exemplary embodiments without departing from the scope of the invention, it is intended that all matter contained in the above description shall be interpreted as illustrative and not in a limiting sense.

What is claimed is:

1. A method of developing a diagnosis and prognosis for an aneurysm in order to determine a procedure for treating the aneurysm, the method comprising:
determining a first diameter of a blood vessel at a first location proximal to an aneurysm;
determining a second diameter of the blood vessel at a second location, said second location incorporating at least a portion of the aneurysm;
calculating the ratio of the second diameter to the first diameter; and
developing a prognosis of the aneurysm based on said ratio.

2. The method of claim 1, wherein the blood vessel is an aorta.

3. The method of claim 2, wherein the aneurysm is an aortic aneurysm.

4. The method of claim 2, wherein the aneurysm is an abdominal aortic aneurysm.

5. The method of claim 1, wherein the first diameter and second diameter are determined using apparatus chosen from ultrasound apparatus, CT apparatus, and MRI apparatus.

6. The method of claim 1, further comprising treating the aneurysm based on said prognosis.

7. The method of claim 6, wherein the treatment is selected from intervention and monitoring said aneurysm.

8. The method of claim 7, wherein the treatment is intervention when said ratio is 2.5 or greater.

9. The method of claim 7, wherein the treatment is monitoring said aneurysm when said ratio is less than 2.5.

10. The method of claim 1, wherein determining the first diameter of the blood vessel and wherein determining the second diameter further comprises directing x-rays or electrical, mechanical, magnetic, or sound waves or pulses from a point exterior to a patient body into the patient body.

11. The method of claim 10, wherein directing x-rays or electrical, mechanical, magnetic, or sound waves or pulses into the patient body further comprises use of an apparatus chosen from an ultrasound apparatus, a CT apparatus, and a MRI apparatus.

12. The method of claim 11, further comprising receiving the x-rays or electrical, mechanical, magnetic, or sound waves or pulses reflected from the blood vessel in the patient body.

13. The method of claim 12, further comprising generating an image of said blood vessel based on the reflected x-rays or electrical, mechanical, magnetic, or sound waves or pulses.

14. The method of claim 13, further comprising reviewing the image and measuring a first diameter and a second diameter of the imaged blood vessel in order to determine the first diameter and the second diameter of the blood vessel.

15. The method of claim 1, wherein measuring the first diameter of the blood vessel and wherein measuring the second diameter further comprises reviewing an image of the blood vessel and measuring a first diameter and a second diameter of the image of the blood vessel in order to determine the first diameter and the second diameter of the blood vessel.

16. An apparatus for use in diagnosing and determining a prognosis for an aneurysm, comprising:
a housing;
a transmitter operatively connected to said housing for directing electrical, mechanical, magnetic, or sound waves or pulses in a direction away from said transmitter and into a patient body;
a receiver operatively connected to said housing for receiving said waves or pulses reflected from a blood vessel in said patient body;
a component operatively connected to said receiver and operable for generating an image of said blood vessel from said reflected waves or pulses; and
a controller adapted to measure a first diameter of said image of said blood vessel at a first location proximal to an aneurysm and measure a second diameter of the image of the blood vessel at a second location, the second location incorporating at least a portion of the aneurysm.

17. The apparatus of claim 16, wherein said controller is further adapted to calculate the ratio of the second diameter to the first diameter.

18. The apparatus of claim 17, wherein said controller is further adapted to display a prognosis of the aneurysm based on said ratio.

19. The apparatus of claim 16, wherein the blood vessel is an aorta.

20. The apparatus of claim 19, wherein the aneurysm is an aortic aneurysm.

21. The apparatus of claim 19, wherein the aneurysm is an abdominal aortic aneurysm.

22. The apparatus of claim 16, wherein the first diameter and second diameter are measured using apparatus chosen from ultrasound apparatus, CT apparatus, and MRI apparatus.

23. The apparatus of claim 18, further comprising treating the aneurysm based on said prognosis.

24. The method of claim 23, wherein the treatment is selected from intervention and monitoring said aneurysm.

25. The apparatus of claim 24, wherein the treatment is intervention when said ratio is 2.5 or greater.

26. The apparatus of claim 24, wherein the treatment is monitoring said aneurysm when said ratio is less than 2.5.

27. The apparatus of claim 16, wherein said component is a transducer.
